(19) United States
(12) Patent Application Publication
(10) Pub. No.: US 2005/0106196 A1
(43) Pub. Date: May 19, 2005

(54) Cosmetic composition containing colloidal particles

(75) Inventors: Guillaume Cassin, Villebon Sur Yvette (FR); Veronique Roger, Bagneux (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/941,844

(22) Filed: Sep. 16, 2004

Related U.S. Application Data
(60) Provisional application No. 60/505,758, filed on Sep. 26, 2003.

(30) Foreign Application Priority Data
Sep. 17, 2003 (FR) 0310915

(51) Int. Cl. A61K 7/021; A61K 7/00

(52) U.S. Cl. 424/401; 424/63

(57) ABSTRACT

The present invention relates to a composition suitable for topical application to the skin, containing a dispersion of colloidal particles, which do not have thickening properties, of at least one inorganic filler—in particular silica—in a physiologically acceptable medium containing at least one volatile oil and at least one nonvolatile oil, the composition being free of cyclopentasiloxane. Advantageously, the weight ratio of volatile oils to nonvolatile oils is between 1:1 and 3:1. It also relates to a method of cosmetic treatment, in particular of an aged skin or a greasy skin, comprising the application to the skin of the abovementioned composition, and the cosmetic uses of this composition, in particular for smoothing wrinkles and fine lines and/or retightening the skin of the face and/or of the neck, for softening or concealing skin imperfections such as fine lines, pores, irregularities of the skin microrelief and pigmented spots, and for mattifying the skin.

### REFERENCE TO PRIOR APPLICATIONS

This application claims priority to U.S. provisional application 60/505,758 filed Sep. 26, 2003, and to French patent application 0310915 filed Sep. 17, 2003, both incorporated herein by reference.

### FIELD OF THE INVENTION

The present invention relates to a composition comprising a dispersion of colloidal particles of at least one inorganic filler—for example silica—in a physiologically acceptable medium comprising at least one volatile oil and one nonvolatile oil, the composition being free of cyclopentasiloxane. Preferably, the composition of the invention is suitable for topical application.

The invention also relates to the cosmetic use of the composition as described above for softening skin imperfections, in particular for concealing and/or smoothing wrinkles and fine lines and/or for mattifying the skin, and to a cosmetic process for care or makeup of the face and/or the neck, in particular of an aged skin or of a greasy skin, comprising the application to the skin of the abovementioned composition.

Additional advantages and other features of the present invention will be set forth in part in the description that follows and in part will become apparent to, those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

During the ageing process, various signs appear on the skin which are very characteristic of this ageing, resulting in particular in a modification of skin structure and functions. The main clinical signs of skin ageing are in particular the appearance of deep wrinkles and fine lines which increase with age.

It is known to treat these signs of ageing using cosmetic or dermatological compositions containing active agents capable of combating ageing, such as α-hydroxy acids, β-hydroxy acids and retinoids. These active agents act on the wrinkles by eliminating the dead cells from the skin and by accelerating the process of cell renewal. However, these active agents have the disadvantage of being effective for the treatment of wrinkles only after a certain period of application. Now, it is increasingly being sought to obtain an immediate effect of the active agents used.

To this end, active agents with a tightening effect have been proposed for the last few years which smooth, immediately after application, the wrinkles and fine lines and contribute towards attenuating the fatigue marks. These compounds act by forming a film, causing retraction of the stratum corneum, the superficial horny layer of the epidermis.

Among these tightening agents are colloidal particles of inorganic fillers such as silica.

While these particles indeed offer a beneficial tightening effect, they have however the disadvantage of lightening the skin when they are formulated in the presence of volatile oils, which adversely affects their use in cosmetic products which should not leave a visible unaesthetic mark on the skin after application.

### SUMMARY OF THE INVENTION

The inventors have now discovered that by formulating inorganic colloidal particles in the presence of a mixture of volatile and nonvolatile oils, preferably in a weight ratio of between 1:1 and 3:1, it is possible to obtain compositions having a tightening effect, making it possible to visibly smooth the wrinkles and fine lines without lightening the skin, and to thereby overcome the disadvantages of the prior art.

U.S. Pat. No. 4,819,825, U.S. Pat. No. 4,777,041 and US2002/0098220 indeed disclose the use of an aqueous dispersion of colloidal or noncolloidal particles of an inorganic filler, in particular silica, for smoothing wrinkles by a tightening effect. The compositions described in these documents are however free of oil.

Other documents describe antiwrinkle compositions comprising colloidal or noncolloidal particles of silica formulated in the presence of oil (FR-2 823 113; FR-2 659 551; WO 02/15873). In these documents, the oils maybe chosen from a long list of oils which may be either volatile or nonvolatile. No example is however given of a formulation simultaneously comprising these two types of oil.

The inventors show herein that the combination of volatile and nonvolatile oils provides a composition which does not lighten on application, while exhibiting a satisfactory tightening effect.

Applications FR 2 838 343 and FR 2 843 024 disclose cosmetic compositions comprising a colloidal dispersion of inorganic particles mixed with volatile and nonvolatile oils. These compositions however systematically contain cyclopentasiloxane.

### DETAILED DESCRIPTION OF THE PREFERRED EMBODIMENTS

One subject of the present invention is therefore a composition that preferably is suitable for topical application to the skin, comprising a dispersion of colloidal particles, which do not have thickening properties, of at least one inorganic filler in a physiologically acceptable medium comprising at least one volatile oil and at least one nonvolatile oil, the composition being free of cyclopentasiloxane.

The expression “colloidal particles, which do not have thickening properties” and “wherein the colloidal particles do not have thickening properties” for the purposes of the present invention is understood to mean a dispersion of particles having a mean diameter in numerical terms of between 0.1 and 100 nm, preferably between 3 and 30 nm, even better between 10 and 15 nm. These particles preserve the abovementioned diameters in the composition containing them, without forming aggregates, and do not therefore have thickening properties in the sense that at a concentration greater than or equal to 15% by weight in any solvent such as water, alcohol or oil, the colloidal particles according to the invention have a viscosity of less than 0.05 Pa.s for a shear rate equal to 10⁻s, the viscosity being measured at 25° C. with the aid of a HAAKE RheoStress RS 150 rheometer in a cone-plane configuration, the cone having a diameter of 60 mm and an angle of 2°.

This particle dispersion thus differs, especially, from dispersions of “fumed” silica particles, which are generally obtained according to a gas-phase process, by hydrolysis of silicon tetrachloride at very high temperature, and which are generally in the form of aggregates, and as such they are incorrectly referred to as “colloidal silica dispersions”. Thus, when the inorganic filler according to the invention consists of silica, it is not fumed.

The dispersion of colloidal particles according to the invention can be prepared according to the so-called “sol-gel” process well known to persons skilled in the art, from corresponding metal salts or alkoxides solubilized in a solvent, such as an alcohol. A hydrolysis reaction is then carried out in order to form an amorphous precipitate. This is then dispersed with an acid or a base according to the desired pH, which leads to the peptization of the precipitate and to crystallization. A crystalline oxide is formed in a solvent in this manner.

Such colloidal suspensions can be for example prepared according to the methods described in J. Colloid Interface Sci., 26, p. 62-69, 1968 for SiO₂, Appli. Opt. 26, 4688, 1987 for TiO₂, Inorg. Chem., 3, 146, 1964 for ZrO₂, Appl. Opt., 27, 3356, 1988 for CaF₂ and MgF₂.

These suspensions are prepared using ionic precursors chosen most often from chlorides, oxychlorides, perchlorates, nitrates, oxynitrates or acetates, or molecular precursors preferably chosen from alkoxides, having the molar formula M(OR)ₙ (M representing a metal, OR an alkoxy radical of 1 to 6 carbon atoms and n representing the valency of the metal). In the methods described above, the precursor is hydrolysed or fluorinated and then polymerized until a finished product, which is insoluble in the chosen solvent, is obtained which is nucleated and called colloidal suspension. In the case of alkoxides, the hydrolysis must be rigorously controlled, given the very hydrophilic character of these organometallic derivatives.

Another method of preparing such products is described in J. LIVAGE et al., “SOL-GEL SYNTHESIS OF METAL OXIDE CLUSTERS AND COLLOIDS” (MAT. RES. SOC. SYNT. PROC., Volume 272, pages 3 to 14).

The inorganic filler used in the composition according to the invention can be chosen in particular from: silica, cerium oxide, zirconium oxide, alumina, silicon carbonate, barium sulphate, calcium sulphate, zinc oxide, titanium dioxide, platinum and mixtures thereof.

Examples of colloidal silicas which can advantageously be used according to the invention include those marketed by the company CATALYSTS & CHEMICALS under the trade names COSMO S-40® and COSMO S-50®. The colloidal particles may, as a variant, consist of a mixed oxide, in particular of a silica-alumina composite filler.

The expression “particles of silica-alumina composite filler” for the purpose of the present invention is understood to mean particles of silicon oxide whose surface has been chemically modified so as to replace at least some of the silicon atoms with aluminium atoms forming at most one monomolecular layer of aluminium. Their aluminium-coated surface portion is generally between 1 and 100%, preferably between 1 and 10%, even better between 4 and 6%.

These particles generally have a zeta potential of less than −20 mV, and more preferably less than −25 mV, at pH 7 and at 25° C., as measured with the aid of a COULTER Scientific Instrument DELSA 440SX apparatus.

They can be prepared in particular as described in patent U.S. Pat. No. 2,892,797, by mixing a silica sol with a sodium aluminate. They are moreover commercially available from the company GRACE under the trade references Ludox AM®, Ludox HSA® and Ludox TMA®.

As indicated above, the composition according to the invention comprises a mixture of volatile and nonvolatile oils which make it possible to avoid the lightening of the skin which is generally observed when the abovementioned particles are applied to the skin.

The expression “oil” is understood to mean for the purposes of the present invention a substance which is insoluble in water (without modification of pH, in an amount of active material of at least 1% by weight at 25° C.), which is liquid at a temperature of less than or equal to 30° C., and which is not considered as a surfactant in at least one of the two following books: McCutcheon's: Emulsifiers and Detergents, International Edition, and International Cosmetic Ingredient Dictionary and Handbook (CTFA).

The expression “volatile oils” is understood to mean specifically oils having at a temperature of 20° C. a vapour pressure greater than 1 mbar. The vapour pressure is defined as the pressure at which a liquid and its vapour are in equilibrium at a given temperature. Particularly useful volatile oils include, inter alia, cyclic and linear silicones containing from 2 to 6 silicon atoms, such as cyclohexasiloxane, dodecamethylpentasiloxane, decamethyltetrasiloxane, butyltrisiloxane and ethyltrisiloxane. It is also possible to use branched hydrocarbons such as for example isododecane and volatile perfluoroalkanes such as dodecafluoropentane and tetradecafluorohexane, sold under the names “PF 5050®” and “PF 5060®” by the company 3M and the perfluoromorpholine derivatives, such as 4-trifluoromethylperfluoromorpholine sold under the name “PF 5052®” by the company 3M.

Useful nonvolatile oils include: hydrocarbon oils of plant origin; linear or branched hydrocarbons of animal, inorganic or synthetic origin; esters of C₈-C₃₀ fatty acids and C₃-C₃₀ monoalcohols; esters of hydroxy acids in which the acid and/or alcohol chain contains from 8 to 29 carbon atoms; pentaerythrityl esters; fluorinated oils; silicone oils; and mixtures thereof.

Useful hydrocarbon oils of plant origin include liquid triglycerides of fatty acids containing from 4 to 10 carbon atoms such as the triglycerides of heptanoic or octanoic acids or, for example, vegetable oils such as sunflower, maize, soybean, gourd, grapeseed, sesame, hazelnut, apricot, macadamia, groundnut, sunflower, castor, avocado, jojoba and shea butter oils, and triglycerides of caprylic/capric acids such as those sold by the company Stearineries Dubois and those sold under the names Miglyol 810, 812 and 818 by the company Dynamit Nobel.

As hydrocarbons of animal origin, there may be mentioned perhydrosqualene, and of inorganic or synthetic origin, there may be mentioned paraffin oils and their derivatives petroleum jelly, polydecenes, hydrogenated polyisobutene such as parleam oil.

Other nonvolatile oils which can be used in the composition according to the invention include esters and in particular esters of C₈-C₃₀ fatty acids and C₃-C₃₀ monoalcohols, such as Purcellin oil, isononyl isononanoate, isopropyl myristate, 2-ethylhexyl palmitate, 2-octyldodecyl stearate, 2-octyldodecyl erucate, isostearyl isostearate; and esters of hydroxyl acids in which the acid and/or alcohol chain contains from 8 to 29 carbon atoms, such as isostearyl lactate, octyl hydroxystearate, octyldodecyl hydroxystearate, diisostearyl malate and triisocetyl citrate; and pentaerythrityl esters such as pentaerythrityl tetraisostearate.

As a variant, the nonvolatile oils used in the composition according to the invention may be linear or branched alcohols having from 8 to 26 carbon atoms, such as octyldodecanol, 2-butyloctanol, 2-hexyldecanol, oleyl alcohol or linoleyl alcohol.

Fluorinated oils which can be used in the composition according to the invention include those described in the document JP-A-2-295912. As fluorinated oils, there may also be mentioned perfluoromethylcyclopentane and perfluoro(1,3-dimethylcyclohexane), sold under the names “FLUTEC PC1®” and “FLUTEC PC3®” by the company BNFL Fluorochemicals; perfluoro(1,2-dimethylcyclobutane); bromoperfluorooctyl sold under the name “FORALKYL®” by the company Atochem; nonafluoromethoxybutane sold under the name “MSX 4518®” by the company 3M and nonafluoroethoxyisobutane.

Useful silicone oils include compounds containing a linear silicone chain, in particular polydimethylsiloxanes containing alkyl, alkoxy or phenyl groups which are pendant or at the silicone chain end, groups having from 2 to 24 carbon atoms; phenylated silicones such as phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes.

The composition according to the invention preferably comprises from 0.5 to 80% by weight, preferably from 1 to 55% by weight, and even better from 1 to 25% by weight (including 2, 4, 6, 8, 12, 14, 16, 18, 20, 22 and 24%) in total of volatile and nonvolatile oils. According to a particularly preferred embodiment of the invention, the weight ratio of volatile oils to nonvolatile oils is between 1:1 and 3:1, including 1.2, 1.4, 1.6, 1.8, 2, 2.2, 2.4, 2.6 and 2.8:1.

The composition according to the invention is preferably suitable for topical application to the skin and therefore generally comprises a physiologically acceptable medium, that is to say compatible with the skin and/or its superficial body growths. Thus, the composition according to the invention preferably has a pH of less than 8, even better of less than or equal to 7, and better still of between 6 and 7.

The quantity of colloidal particles present in the composition may vary to a large degree according to the desired effect. By way of example, these particles may represent from 0.01 to 15% by weight, and preferably from 1 to 10% by weight, including 1, 2, 3, 4, 5, 6, 7, 8, and 9% by weight, relative to the total weight of the composition.

The composition according to the invention may be provided in any form, including galenic forms conventionally used for topical application and in particular in the form of dispersions of the lotion or serum type, of emulsions of liquid or semiliquid consistency of the milk type, which are obtained by dispersing a fatty phase in an aqueous phase (O/W) or conversely (W/O), or of suspensions or emulsions of soft, semisolid or solid consistency of the cream or gel type, of alternatively of multiple emulsions (W/O/W of O/W/O), of microemulsions, of vesicular dispersions of the ionic and/or nonionic type, or of wax/aqueous phase dispersions. These compositions are prepared according to the customary methods.

According to a preferred embodiment of the invention, the composition is provided in the form of an O/W, W/O or W/O/W emulsion.

This composition may be a product for care of the face and/or neck. Alternatively it may be a makeup product such as a foundation.

The composition according to the invention may additionally contain various adjuvants, for example those commonly used in the cosmetics field, such as emulsifiers, fillers, preservatives, sequestrants, colorants, perfumes, thickeners and gelling agents. The quantities of these various adjuvants and their nature will preferably be chosen so as not to adversely affect the properties of the composition according to the invention, that is to say that they will not negatively affect the skin-tightening capacity of this composition and will not leave a whitish deposit thereon upon application.

According to an advantageous embodiment, the composition according to the invention is free of glycerine. The inventors have indeed observed that the tightening effect of the compositions according to the invention was improved in the absence of glycerine.

The inventors have discovered that the composition according to the invention is endowed with antiwrinkle properties by the tightening effect, and so-called “soft-focus” optical properties which make it possible to envisage its use to mattify the skin, homogenize the complexion and soften the skin imperfections, while exhibiting a very good cosmetic feel.

One subject of the invention is therefore also a cosmetic process for care or makeup of the face and/or neck, in particular of an aged skin or of a greasy skin, comprising the application to the skin of the abovementioned composition. Its subject is also the cosmetic use of the composition defined above for smoothing wrinkles and fine lines and for retightening the skin of the face and/or of the neck.

Its subject is also the cosmetic use of the composition as defined above for softening or concealing skin imperfections such as fine lines, pores, irregularities of the skin microrelief and pigmented spots.

Its subject is also the cosmetic use of the composition as defined above for mattifying the skin.

To enhance the antiage effects of the composition according to the invention, the latter may contain at least one compound chosen from: desquamating and/or moisturizing agents; depigmenting or propigmenting agents; antiglycation agents; agents stimulating the synthesis of dermal or epidermal macromolecules and/or preventing their degradation; agents stimulating the proliferation of the fibroblasts and/or the keratinocytes or stimulating the differentiation of the keratinocytes; dermodecontracting agents; antipollution and/or anti-free radical agents; slimming agents; agents acting on the microcirculation; agents acting on the energy metabolism of the cells, and mixtures thereof.

Thus, the composition according to the invention may contain in particular at least one active agent chosen from: α-hydroxy acids; salicylic acid and its derivatives such as 5-(n-octanoyl)salicylic acid; HEPES; L-2-oxothiazolidine-4-carboxylic acid; O-octanoyl-6-D-maltose; disodium salt of methylglycinediacetic acid; ceramides; steroids such as diosgenin and DHEA derivatives; niacinamide; ascorbic acid and its derivatives; bilberry extracts; retinoids and in particular retinol and its esters; polypeptides and their acylated derivatives; phytohormones; Saccharomyces cerevisiae yeast extracts; algal extracts; extracts of Vitreoscilla filiformis; soybean, lupin, maize and/or pea extracts; alverine and its salts, in particular alverine citrate; resveratrol; carotenoids and in particular lycopene; coenzyme Q10 or ubiquinone; xanthines and in particular caffeine and natural extracts containing it; extracts of butcher's broom and of horse chestnut; and mixtures thereof, without this list being limiting.

The composition according to the invention may additionally contain at least one UVA- and/or UVB-screening agent. The sunscreens may be chosen from organic screening agents, inorganic screening agents and mixtures thereof.

In the case where the composition according to the invention is used in the treatment of greasy skins, the composition according to the invention may comprise at least one active agent chosen from: desquamating agents; antiseborrhoeic agents, and in particular 5-reductase inhibitors; soothing agents; antibacterial agents; and mixtures thereof.

These active agents may be chosen in particular from retinoids and in particular retinol; zinc salts such as zinc gluconate; an extract of Laminaria saccharina; triclosan; phenoxyethanol; octoxyglycerine; octanoylglycerine; caprylyl glycol; azelaic acid, α-hydroxy acids such as lactic or glycolic acids; β-hydroxy acids, in particular salicylic acid and its derivatives such as 5-(n-octanoyl)salicylic acid; ursolic acid; panthenol; and octopirox.

Besides the colloidal particles of inorganic filler, the composition according to the invention may also comprise various fillers that absorb sebum and/or that have an optical effect.

For the purposes of the invention, the expression “filler with an optical effect” denotes a filler with a refractive index of less than or equal to 2.2, especially less than or equal to 2 and in particular less than or equal to 1.8, preferably ranging from 1.3 to 1.6.

More particularly, these fillers with an optical effect may be chosen, for example, from:
polyamide (Nylon®) powders, such as, for example, the particles of Nylon 12 of the Orgasol type from Atofina, with a mean size of 10 microns and a refractive index of 1.54;
silica powders such as, for example, the Silica beads SB 150 from Miyoshi, with a mean size of 5 microns and a refractive index of 1.45;
polytetrafluoroethylene powders, for instance PTFE Ceridust 9205F from Clariant, with a mean size of 8 microns and a refractive index of 1.36;
silicone resin powders, for instance the silicon resin Tospearl 145A from GE Silicone, with a mean size of 4.5 microns and a refractive index of 1.41;
powders of acrylic copolymers, especially of polymethyl (meth)acrylate, for instance the PMMA particles Jurymer MBI from Nihon Junyoki, with a mean size of 8 microns and a refractive index of 1.49;
wax powders, for instance the paraffin wax particles Microease 114S of micropowders with a mean size of 7 microns and a refractive index of 1.54;
polyethylene powders, especially comprising at least one ethylene/acrylic acid copolymer, and in particular consisting of ethylene/acrylic acid copolymers, for instance the particles Flobeads EA 209 from Sumitomo (mean size of 10 microns and a refractive index of 1.48);
powders of elastomeric crosslinked organopolysiloxane, coated with silicone resin, especially with silsesquioxane resin, as described, for example, in patent U.S. Pat. No. 5,538,793. Such elastomeric powders are sold under the names “KSP-100”, “KSP-101”, “KSP-102”, “KSP-103”, “KSP-104” and KSP-105” by the company Shin-Etsu;
talc/titanium dioxide/alumina/silica composite powders, for instance those sold under the name Coverleaf AR-80 by the company Catalyst & Chemicals; and
mixtures thereof.

The expression “sebum-absorbing compound” means a compound capable of absorbing and/or adsorbing sebum, having a sebum uptake generally of greater than or equal to 1 ml/g, especially greater than or equal to 2 ml/g and in particular greater than or equal to 3 ml/g.

The particles of sebum-absorbing and/or sebum-adsorbing compound may be of inorganic or organic origin.

More specifically, this compound may be chosen from fumed silica, polyamide (Nylon®) powders, powders of acrylic polymers, especially of polymethyl methacrylate, of polymethyl methacrylate/ethylene glycol dimethacrylate, of polyallyl methacrylate/ethylene glycol dimethacrylate and of ethylene glycol dimethacrylate/lauryl methacrylate copolymer, and mixtures thereof.

The composition according to the invention may also contain various additional fillers of inorganic or organic origin. They may be of any form, especially of platelet, spherical or oblong form, irrespective of their crystallographic form (for example lamellar, cubic, hexagonal, orthorhombic, etc.).

Among these additional fillers that may be used in the composition according to the invention, mention may be made especially of talc, mica, kaolin, poly-β-alanine and polyethylene, lauroyllysine, starch, boron nitride, precipitated calcium carbonate, magnesium carbonate and hydrocarbonate, barium sulphate, hydroxyapatite, glass or ceramic microcapsules, and metal soaps derived from organic carboxylic acids containing from 8 to 22 carbon atoms and especially from 12 to 18 carbon atoms, for example zinc stearate, magnesium stearate, lithium stearate, zinc laurate or magnesium myristate.

The composition according to the invention may also advantageously contain at least one pulverulent dyestuff, which may be chosen from the pigments and nacres usually used in cosmetic and/or dermatological compositions.

The pigments may be white or coloured, inorganic and/or organic, and coated or uncoated. Among the inorganic pigments that may especially be mentioned are titanium dioxide, optionally surface-treated, zirconium oxide or cerium oxide, and also iron oxide, chromium oxide, manganese violet, ultramarine blue, chromium hydrate and ferric blue. Among the organic pigments that may especially be mentioned are carbon black, pigments of D & C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the abovementioned type, and also nacreous pigments based on bismuth oxychloride.

The invention will now be illustrated by the following nonlimiting examples. In these examples, the quantities are indicated as a percentage by weight and the compounds are generally identified according to the CTFA nomenclature.

### EXAMPLES

### Example 1

### Cosmetic Composition in the Form of an O/W Emulsion

| | |
|---|---|
| Phase A | |
| Dimyristyl Tartrate (and) Cetearyl Alcohol (and) | 1.50 g |
| C₁₂₋₁₅ Pareth-7 (and) PPG-25-Laureth-25 | |
| Glyceryl Stearate (and) PEG-100 stearate | 2.00 g |
| Stearyl alcohol | 1.00 g |
| Cyclohexasiloxane | 7.50 g |
| Hydrogenated polyisobutene | 2.50 g |
| Phase B | |
| Preservatives | 1.00 g |
| Ammonium polyacryloyldimethyl taurate | 0.40 g |
| Water | 66.75 g |
| Xanthan gum | 0.20 g |
| Pentasodium ethylenediaminetetramethylene- | 0.05 g |
| phosphonate | |
| Phase C | |
| Colloidal dispersion of particles of silica | 17.10 g |
| (COSMO S-40 from Catalysts & Chemicals) | |

Procedure:

The above composition was prepared in the following manner: phase B was heated to about 75° C. and ammonium polyacryloyldimethyl taurate was incorporated therein and then the mixture was stirred until a homogeneous gel was obtained. Separately, phase A was heated to about 75° C. The emulsion was then prepared by incorporating phase A into phase B, and then phase C was incorporated at 40-45° C. while the stirring is maintained until complete cooling is obtained.

### Example 2 (Comparative)

### Cosmetic Composition in the Form of an O/W Emulsion

| | |
|---|---|
| Phase A | |
| Dimyristyl Tartrate (and) Cetearyl Alcohol (and) | 1.50 g |
| C₁₂₋₁₅ Pareth-7 (and) PPG-25-Laureth-25 | |
| Glyceryl Stearate (and) PEG-100 stearate | 2.00 g |
| Stearyl alcohol | 1.00 g |
| Cyclohexasiloxane | 10.00 g |
| Phase B | |
| Preservatives | 1.00 g |
| Ammonium polyacryloyldimethyl taurate | 0.40 g |
| Water | 66.75 g |
| Xanthan gum | 0.20 g |
| Pentasodium ethylenediaminetetramethylene- | 0.05 g |
| phosphonate | |
| Phase C | |
| Colloidal dispersion of particles of silica | 17.10 g |
| (COSMO S-40 from Catalysts & Chemicals) | |

The above composition was prepared as described in Example 1.

### Example 3

### Evaluation of the Lightening Effect

The compositions of Examples 1 and 2 were tested on an area of the back of the hand at the rate of about 5 mg/cm². Thirty minutes after the application, a photograph was taken of the treated areas. The composition of Example 1 according to the invention does not leave any white deposit on the skin after application (right hand), unlike the composition of comparative Example 2 (left hand).

### Example 4

### Evaluation of the Optical Properties In Vivo

The antiwrinkle, “soft-focus” and mattifying effects of the composition of Example 1 were evaluated on a panel of 12 women. It was observed that this composition reduced the visibility of the skin microrelief by slimming down the grain of the skin and mattifying the glints instantly, in a significant manner up to 4 hours after the application. Simultaneously, the complexion appeared more unified and the wrinkles and fine lines softened. No lightening of the skin was demonstrated.

### Example 5

### Evaluation of the Tightening Effect In Vivo

The composition of Example 1 was applied to a panel of women aged from 40 to 60 years, having wrinkles and fine lines on the contour of the eye (studied by bilateral photographs). A significant effect of smoothing of the fine lines which exist under the eye and of the crow's-foot wrinkles was observed after application of the composition, without having observed any problem of unaesthetic lightening.

The above written description of the invention provides a manner and process of making and using it such that any person skilled in this art is enabled to make and use the same, this enablement being provided in particular for the subject matter of the appended claims, which make up a part of the original description and including a composition comprising a dispersion of colloidal particles of at least one inorganic filler in a physiologically acceptable medium comprising at least one volatile oil and at least one nonvolatile oil, wherein the composition is free of cyclopentasiloxane, and wherein the colloidal particles do not have thickening properties.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

Preferably, the amounts of invention composition used in the methods of the invention are amounts effective to accomplish the stated goals of the methods, for example, effective amounts to care or makeup of the face and/or the neck, effective amounts for concealing skin imperfections such as fine lines, pores, irregularities of the skin microrelief and pigmented spots, effective amounts for mattifying the skin, effective amounts for smoothing wrinkles and fine lines and/or retightening the skin of the face and/or of the neck, etc. Such amounts are determinable by one of ordinary skill in this art in view of the present disclosure without undue experimentation.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising a dispersion of colloidal particles of at least one inorganic filler in a physiologically acceptable medium comprising at least one volatile oil and at least one nonvolatile oil, wherein the composition is free of cyclopentasiloxane, and wherein the colloidal particles do not have thickening properties.

2. The composition according to claim 1, wherein the inorganic filler is selected from the group consisting of silica, cerium oxide, zirconium oxide, alumina, silicon carbonate, barium sulphate, calcium sulphate, zinc oxide, titanium dioxide, platinum and mixtures thereof 3. The composition according to claim 1, wherein the inorganic filler is non-fumed silica.

4. The composition according to claim 1, wherein the inorganic filler is a mixed oxide.

5. The composition according to claim 4, wherein the inorganic filler is a silica-alumina composite filler.

6. The composition according to claim 5, wherein the surface of the particles which is covered with aluminium is between 4 and 6%.

7. The composition according to claim 5, wherein the particles have a zeta potential of less than −25 mV, at pH 7 and at 25° C.

8. The composition according to claim 1, wherein the particles have a mean diameter in numerical terms of between 0.1 and 100 nm.

9. The composition according to claim 8, wherein the particles have a mean diameter in numerical terms of between 3 and 30 nm.

10. The composition according to claim 1, wherein the composition has a pH of less than or equal to 7.

11. The composition according to claim 10, wherein the composition has a pH of between 6 and 7.

12. The composition according to claim 1, comprising from 1 to 10% by weight of colloidal particles, relative to the weight of the composition.

13. The composition according to claim 1, wherein said composition is free of glycerine.

14. The composition according to claim 1, comprising at least one nonvolatile oil selected from the group consisting of hydrocarbon oils of plant origin; hydrocarbons of animal, inorganic or synthetic origin; esters of C₈-C₃₀ fatty acids and C₃-C₃₀ monoalcohols; esters of hydroxy acids in which the acid and/or alcohol chain contains from 8 to 29 carbon atoms; pentaerythrityl esters; linear or branched alcohols having from 8 to 26 carbon atoms; fluorinated oils; silicone oils;
and mixtures thereof.

15. The composition according to claim 1, comprising at least one volatile oil selected from the group consisting of: cyclic silicones, linear silicones containing from 2 to 6 silicon atoms, branched hydrocarbons, perfluoroalkanes and perfluoromorpholine derivatives and mixtures thereof.

16. The composition according to claim 1, comprising from 1 to 25% by weight in total of volatile and nonvolatile oils.

17. The composition according to claim 1, wherein the weight ratio of volatile oils to nonvolatile oils is 1:1 and 3:1.

18. The composition according to claim 1, wherein said composition is in the form of an O/W, W/O or W/O/W emulsion.

19. A process for care or makeup of the face and/or the neck, comprising the application to the skin of the composition according to claim 1.

20. A process for softening or concealing skin imperfections such as fine lines, pores, irregularities of the skin microrelief and pigmented spots, comprising the application of the composition according to claim 1 to skin in need thereof.

21. A process for mattifying the skin, comprising the application of the composition according to claim 1 to skin in need thereof.

22. A process for smoothing wrinkles and fine lines and/or retightening the skin of the face and/or of the neck, comprising the application of the composition according to claim 1 to skin in need thereof.
